(19) United States
(12) Patent Application Publication
(10) Pub. No.: US 2023/0405323 A1
(43) Pub. Date: Dec. 21, 2023

(54) SELECTIVE STIMULATION OF GROUPS OF NEURAL ELEMENTS WITHIN A REGION OF THE BRAIN

(71) Applicants: Case Western Reserve University, Cleveland, OH (US); The UAB Research Foundation, Birmingham, AL (US)

(72) Inventors: Michael Moffitt, Solon, OH (US); Harrison Walker, Birmingham, AL (US)

(21) Appl. No.: 18/337,705

(22) Filed: Jun. 20, 2023

Related U.S. Application Data
(60) Provisional application No. 63/353,727, filed on Jun. 20, 2022.

(51) Int. Cl. A61N 1/36

(52) U.S. Cl. CPC A61N 1/36067; A61N 1/36171

(57) ABSTRACT

Symptoms of maladies originating in the same region of a brain can be treated with selective multifrequency stimulation. A first stimulation at a first frequency and a second stimulation at a second frequency can be generated by a signal generator. The first stimulation can be applied to a first location in the brain region to trigger an excitation period for a first set of neural elements in the brain region to treat at least one symptom of the malady. The second stimulation can be applied, after a time to ensure the first set of neural elements has entered a refractory period, to a second location in the brain region to trigger an excitation period for a second set of neural elements in the brain region to treat the at least one symptom and/or at least another symptom. The first set of neural elements is not excited by the second stimulation.

### CROSS-REFERENCE TO RELATED APPLICATIONS

This application claims the benefit of U.S. Provisional Application Ser. No. 63/353,727, filed 20 Jun. 2022, entitled “SELECTIVE STIMULATION OF GROUPS OF NEURAL ELEMENTS WITHIN A REGION OF THE BRAIN”. The entirety of this provisional application is incorporated by reference for all purposes.

### TECHNICAL FIELD

The present disclosure relates generally to stimulation of neural elements within a region of a brain (or other elements of the central or peripheral nervous systems) and, more specifically, to systems and methods for selective stimulation of groups of neural elements within the region of the brain (or other elements of the central or peripheral nervous systems) utilizing two independently programmed stimulation fields.

### BACKGROUND

Parkinson's disease affects nearly one million people in the United States. Sufferers can experience both appendicular symptoms, like tremor, rigidity, and bradykinesia of the extremities, and axial symptoms, like balance and gait issues. While the underlying cause of Parkinson's disease cannot currently be cured, the appendicular and axial symptoms can be treated and mitigated. Normally, at least one of the appendicular symptoms can be treated by stimulation of a first group of neural elements at a first frequency, while at least one of the axial symptoms can be treated by stimulation of a second group of neural elements at a second frequency. One problem inherent with treating both the appendicular and axial symptoms is that the appendicular and axial symptoms may each be regulated by neural elements in the same, overlapping, or adjacent neighborhoods in the subthalamic region of the brain that are in a certain proximity to one another. With neural elements within certain proximities of each other, the influence of both stimulation fields on the activity of a portion of the neural elements renders true frequency independent stimulation not achievable or unrealistically cumbersome.

### SUMMARY

The present disclosure relates to systems and methods for selective stimulation of a region of the brain using multiple frequencies to treat multiple neurologically based symptoms of a malady, such as cardinal and axial symptoms of Parkinson's disease (including different neural elements that may be within close proximity to, adjacent to, or partially overlapping each other). The selective stimulation utilizes two independently programmed stimulation fields (e.g., at different frequencies or with different patterns) to stimulate the different neural elements in a non-cumbersome manner.

In an aspect, the present disclosure can include a method for treating symptoms of a malady. A first stimulation at a first frequency and a second stimulation at a second frequency can be generated. The first stimulation can be applied to a first location in a region of a brain of a patient to trigger an excitation period for a first set of neural elements in the region of the brain to treat at least one symptom of the malady. After a time to ensure the first set of neural elements has entered a refractory period, the second stimulation can be applied to a second location in the region of the brain to trigger an excitation period for a second set of neural elements in the region of the brain to treat at least another symptom of the malady, where the first set of neural elements is not excited by the second stimulation.

In another aspect, the present disclosure can include a system that can treat symptoms of the malady. The system includes a signal generator configured to generate a first stimulation at a first frequency and a second stimulation at a second frequency. The system also includes at least one DBS lead configured to be positioned within a region of the patient's brain, where the region comprises a first set of neural elements and a second set of neural elements. The at least one DBS lead can be configured to: apply the first stimulation to a first location in the region of the brain to trigger an excitation period of the first set of neural elements to treat at least one symptom of the malady, and after a time to ensure the first set of neural elements enters a refractory period, apply the second stimulation to a second location in the region of the brain to trigger an excitation period for only the second set of neural elements to treat at least another symptom of the malady, where the first set of neural elements is not excited by the second stimulation. In some instances, the excitation of the second set of neural elements can provide an additive effect to the treatment of the same symptoms treated by the first frequency.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a system that can treat multiple symptoms of a malady with selective stimulation that allows for independent programming of required stimulation fields in a manner that is not cumbersome in accordance with an aspect of the present disclosure;

FIG. 2 is a diagram illustrating a traditional single pulse stimulation in comparison to a dual pulse stimulation that can be delivered by the system of FIG. 1;

FIG. 3 includes diagrams illustrating examples of different first and second stimulations that can be delivered by the system of FIG. 1;

FIG. 4 includes illustrations of example first and second stimulation pulses that can be independently programmed and delivered by the system of FIG. 1; and

FIG. 5 is a process flow diagram illustrating a method for treating at least one symptom of a malady of a patient with selective stimulation that allows for independent programming of required stimulation fields in a manner that is not cumbersome in accordance with another aspect of the present disclosure.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “stimulation” can refer to delivery of a signal (e.g., an electrical signal) to activate conduction within at least one neural element. A stimulation signal can have either a positive or a negative polarity. Applying a stimulation to one or more neural elements, can result in the one or more neural elements undergoing an excitation period and a refractory period.

As used herein, the term “excitation period” can refer to the time period when a neural element is in a state of depolarization until an action potential is generated.

As used herein, the term “refractory period” can refer to the period after an action potential is generated in a neural element when the ion channels in the cellular membrane of the neural element reach a state in which a subsequent action potential cannot be generated (absolute refractory period) or require a stronger stimulus (relative refractory period).

As used herein, the term “absolute refractory period” can refer to the period immediately following the firing of a neural element when the neural element cannot be activated, regardless of the strength of the stimulus applied. The absolute refractory period starts immediately after the initiation of the action potential and lasts for a period of time after the peak of the action potential and into the neural elements' recovery toward steady-state.

As used herein, the term “relative refractory period” can refer to the period immediately following the absolute refractory period when partial repolarization has occurred and a greater than normally needed stimulus is needed in order to elicit a second action potential.

As used herein, the term “neural element” can refer to any part of the brain that can conduct an electrical stimulation. A neural element can be, for example, neurons, nerve fibers, neural cells, axons of passage, axon terminals, or the like, in the brain. Neural elements can have various sizes, orientations, and electrical stimulation response dynamics. Populations of neural elements can be grouped together based on at least one of location and electrical stimulation response characteristics (e.g., how the neural elements respond to stimulations with different properties (e.g., polarity/field orientation, pulse width, frequency, duty cycle/burst/patterns, or the like).

As used herein, the terms “region of the brain” or “brain region” can refer to an anatomical region of the brain defined by anatomical landmarks, such as a particular lobe, a structure, a nucleus, a tract, a covering, etc. or a region of the brain defined by man-made markers (electrode, lead, etc.).

As used herein, the term “proximal” can refer to being near (not in direct contact) or in direct contact.

As used herein, the term “patient” can refer to a mammal suffering from a malady.

As used herein, the term “malady” can refer to a disease, disorder, illness, or injury that has one or more symptoms. One example malady is Parkinson's disease. Other examples of maladies are, for example (not exclusive): vascular perfusion, gastrointestinal dysfunction, inflammation, fatigue in functional electrical stimulation, overactive bladder, fecal Incontinence, Alzheimer's disease, depression, arthritis, Crohn's disease, movement disorders in addition to Parkinson's disease (Dystonia, Essential Tremor, Epilepsy), indications of these maladies, or the like.

As used herein, the term “symptom” can refer to a physical or mental feature which is regarded as indicating a condition of a malady. Non-limiting examples of symptoms of Parkinson's disease include tremor, bradykinesia (e.g., slowed movement), rigid muscles, axial problems, gait problems, balance problems, impaired posture (e.g., a stoop), loss of automatic movements. Different symptoms can be ameliorated and/or treated with stimulation of different sets of neural elements.

As used herein, the term “deep brain stimulation (DBS) lead” can refer to one or more electrode contacts connected by a wire that conveys a stimulation signal from a source to the one or more electrode contacts and is implanted in certain deep brain structures (such as the thalamus, the globus pallidus, the subthalamic nucleus, or the like) for deep brain stimulation applications. Where a DBS lead is described herein, the DBS lead may be more than one DBS lead or may be any grouping of one or more electrodes implantable within or on any portion of the brain.

### II. Overview

Stimulations of different populations of neural elements (e.g., two populations that are proximal, adjacent, or overlapping) can be programmed with different frequencies in a manner that is not cumbersome so that the stimulations are independent and selective using refractory shadowing. After an action potential has been generated in a neural element, the ion channels in the membrane of the neural element reach a state in which a subsequent action potential either cannot be generated (an absolute refractory period) or requires a stronger stimulus to be generated (relative refractory period). Refractory shadowing builds on the fact that different populations of neural elements within the brain respond to different stimulation properties (e.g., polarity, pulse-width, amplitude, frequency, patterns, etc.). When a first population of neural elements in a region is already in a refractory period from a stimulation it cannot respond to the second stimulation but the second population, which did not respond to the first stimulation can respond to the second stimulation. When used in conjunction with at least two stimulation frequencies or patterns, refractory shadowing can achieve independent stimulation. Different frequencies or patterns applied to at least two neighboring neural element populations can circumvent the traditional need for a minimum amount of spatial distance between the two populations of neural elements to be stimulated so that each stimulation is independent and separate. Further, the refractory shadowing method reduces or eliminates the possibility that some neural elements are excited by both the first and second stimulations resulting in an undesired cumulative excitation effect, which occurs when using traditional methods and there is not an adequate distance between the spatial regions.

Treatment of symptoms of Parkinson's diseases and specific application to neural elements of the brain are described herein in detail, however it should be understood that the systems and methods described herein can be applied to treat one or more neurologically based symptoms of other maladies (caused by one or more nerves of the central nervous system and/or peripheral nerves). The treatment can be via selective stimulation using multiple frequencies.

### III. Systems

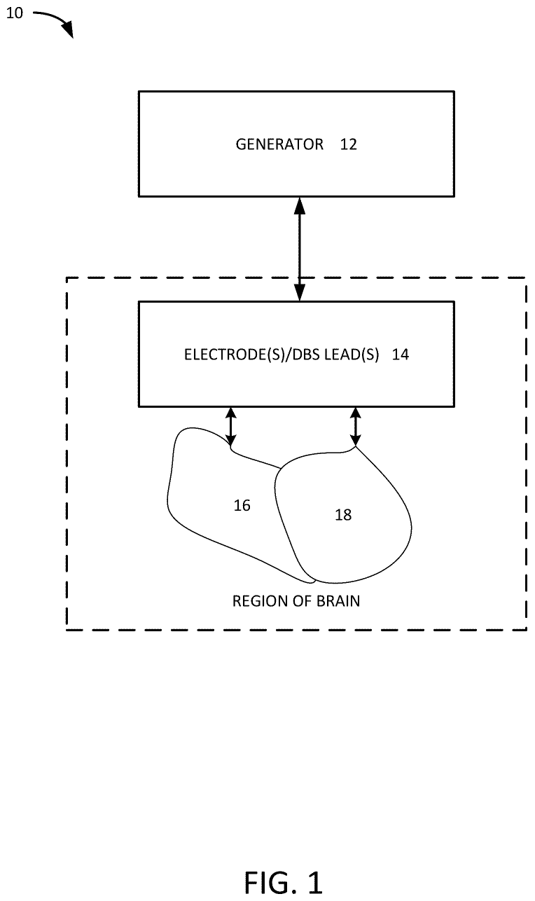

An aspect of the present disclosure can include a system 10 (FIG. 1) that can independently and selectively stimulate different populations of neural elements at multiple frequencies using refractory shadowing. The system 10 can simultaneously treat symptoms of maladies that originate in the same region of a patient's brain. For example, Parkinson's disease can cause multiple symptoms, sometimes called cardinal and axial symptoms, that may be treatable by targeting neural elements within a neighborhood within the subthalamic region that may be adjacent or overlapping in a region of the brain with different types of stimulations. For example, cardinal symptoms of Parkinson's diseases that can be treated with a first stimulation at a first frequency (e.g., 100 Hz or greater, 130 Hz or higher, or 185 Hz and higher) can include tremor, rigidity, and bradykinesia, while axial symptoms of Parkinson's disease that can be treated with a second stimulation at a second frequency (e.g., 90 Hz or lower, 75 Hz or lower, 65 Hz or lower, or 60 Hz or lower) can include balance and gait. It should be understood that the system 10 can be used to treat other maladies.

The system 10 includes a signal generator 12 in electrical communication with one or more electrodes, which can be part of a deep brain stimulation (DBS) lead 14 having one or more electrical contacts implantable in the brain (which also may be referred to as one or more electrodes). It should be noted that while a DBS lead is illustrated and described herein, it is not required and any grouping of one or more electrodes can deliver the stimulations. The signal generator 12, in some instances, can be directly programmable (e.g., including at least a user interface, a non-transitory memory, and a processor, not shown) and/or programmable via a remote device (not shown), such as a mobile device or computer, that has at least a user interface, a non-transitory memory, and a processor. In other words, the signal generator 12 can be programmed via a wired connection to the signal generator 12 (e.g., a keyboard or other input device) or a wireless connection to the signal generator 12 (e.g., via a remote device over the internet or an intranet). The signal generator 12 can generate a first stimulation at a first frequency and a second stimulation at a second frequency and send the stimulations to the at least one DBS lead 14. The signal generator 12 may generate the first stimulation at least twice before generating the second stimulation. The first frequency can be an integer multiple of the second frequency. In one example, the second frequency can be 100 Hz or less, 90 Hz or less, 75 Hz or less, 65 Hz or less, or 60 Hz or less, and the first frequency can be 100 Hz or higher, 130 Hz or higher, or 185 Hz or higher. In some examples, the first frequency can be an integer multiple of the second frequency. The signal generator 12 can modulate at least one of a pattern, a polarity, an amplitude, the first frequency or the second frequency, a pulse width, and a stimulation location of the first stimulation and/or the second stimulation in response to receiving feedback (e.g., user evaluation input, sensed physiological signals, signals from an accelerometer, an IMU, a gyroscope, or the like). Modulations can be based on the application mechanism. For example, using DBS lead(s) refractory periods tend to end approximately 500 μs after a pulse of the first stimulation is applied, so the second stimulation should be delivered during that time window, but not so close together as to deliver only a single large pulse (e.g., after 50 μs, 100 μs, or the like, but before the absolute refractory period ends approximately 500 μs after the first stimulation pulse ends). Pulses after this time can be delivered during a relative refractory period with reduced selectivity (until the excitable membranes return to a baseline state).

The at least one DBS lead 14 can be positioned in a region of the patient's brain that comprises a first set of neural elements 16 and a second set of neural elements 18. The region of the brain may be the subthalamic neighborhood. The at least one DBS lead 14 may be previously implanted in the brain of the patient. The DBS lead 14 can have at least one contact configured to deliver the first stimulation to a first location and at least one contact configured to deliver the second stimulation to a second location. The DBS lead 14 can also having one contact configured to deliver the first stimulation to the first location and the second stimulation to the second location when the first and second locations are proximal or at the same (identical) location. The first set of neural elements 16 can have an effect on at least one symptom of a malady (e.g., can be a neural origin of the symptom, can disrupt the symptom, or the like). The second set of neural elements 18 can have an effect on the symptom and/or at least another symptom of a malady (e.g., can be a neural origin of the symptom or other symptom, can disrupt the symptom or the other symptom, or the like). The first set of neural elements 16 and the second set of neural elements 18 can be proximal to each other, contiguous, and/or at least partially overlapping.

The at least one DBS lead 14 can apply the first stimulation to the first location in the region of the brain to trigger an excitation period for the first set of neural elements 16 to treat at least one symptom of a malady. After a time to ensure the first set of neural elements 16 has entered a refractory period (absolute or relative), the at least one DBS lead 14 can apply the second stimulation to a second location in the region of the brain to trigger an excitation period for only the second set of neural elements 18 to treat at least the other symptom of the malady. The first set of neural elements 16 is not excited by the second stimulation when the first set of neural elements is in an absolute refractory period and has reduced excitability during a relative refractory period. The first location can include a portion of the first set of neural elements 16 or can be in electrical communication with the first set of neural elements. The second location can include a portion of the second set of neural elements 18 or can be in electrical communication with the second set of neural elements. In electrical communication refers to neural elements that are affected by stimulation of the first and/or second location even if not at those locations. In an example where the malady is Parkinson's disease, the at least one symptom can be a appendicular symptom such as tremor, rigidity, and/or bradykinesia of an extremity, and the at least the other symptom can be an axial symptom such as a gait problem and/or a balance problem.

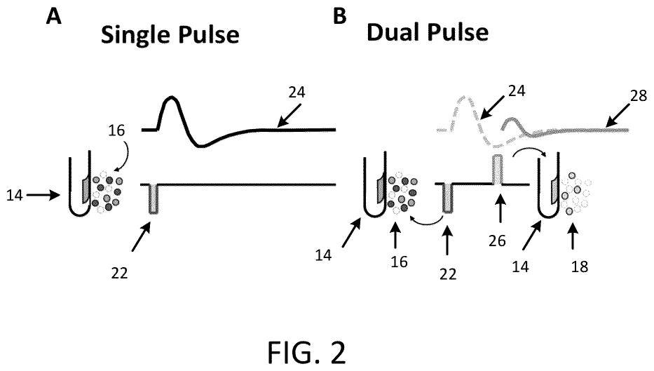

FIG. 2 illustrates in panel A what happens when a traditional single pulse at a given frequency is applied to neural elements in comparison to what happens in panel B when a dual pulse is applied at given frequencies to neural elements. First, with respect FIG. 2 panel A the prior art single pulse example, a DBS lead 14 is positioned within a region of a brain in electrical communication with the first set of neural elements 16. A single pulse, the first stimulation 22, is applied through the DBS lead 14. The line 24 represents the action potential generated in the first set of neural elements 16 after the first stimulation 22 is applied. First an excitation period (the “peak”) occurs and then one or more refractory periods occur (the “valley”) (it should be noted that one nerve has one refractory period, but multiple nerves can have an equal number of refractory periods as the number of nerves) before the conductivity of the first set of neural elements returns to normal. Only the first set of neural elements 16 are activated by the single pulse of the first stimulation 22.

Second, with respect to FIG. 2, panel B, the dual pulse example, one or more DBS leads 14 (here shown as one electrode on one lead but can be any number electrodes and leads) are positioned within a region of a brain in electrical communication with the first set of neural elements 16 and the second set of neural elements 18. The first set of neural elements 16 can be stimulated by a first stimulation pulse 22 of a given polarity and the second set of neural elements 18 can be stimulated by a second stimulation pulse 26 of an opposite polarity. While shown as opposite polarities the first stimulation 22 and the second stimulation 26 may also have the same polarity if other characteristics differ (e.g., frequency, amplitude, pulse width, stimulation location (e.g., through active electrode choice), etc.) In the dual pulse example, the first stimulation 22 and the second stimulation 26 are applied to first and second sets of neural elements 16, 18, respectively, at different times. The first stimulation 22 is applied first to generate the action potential 24 as described with the single pulse example. Then, when the first set of neural elements 16 have entered a refractory period the second stimulation 26 is applied. The second stimulation 26 causes an action potential 28 in the second set of neural elements 18 but does not excite the first set of neural elements 16. The action potential 28 in the second set of neural elements 18 also includes an excitation period and one or more refractory periods. The type of refractory period the neural elements undergo can be based on at least one of the time elapsed after the action potential, and/or the type of neural elements stimulated. The second stimulation 26 has an opposite polarity to the first stimulation 22 to effect different sets of neural elements. Thus, the first set of neural elements 16 and the second set of neural elements 18 can be selectively stimulated with stimulations of different characteristics.

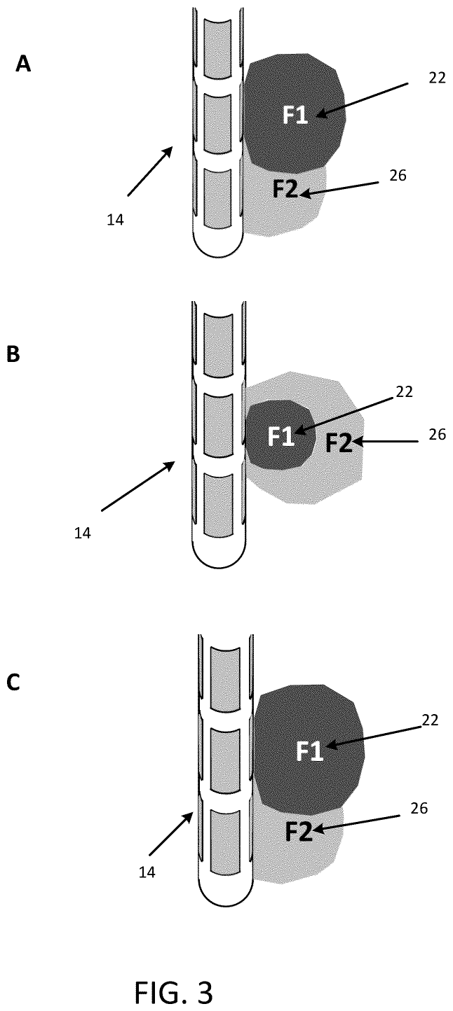

FIG. 3 shows example illustrations of the first stimulation 22 and the second stimulation 26, delivered by contacts of a DBS lead 14, each illustration details example locations and frequencies. While not illustrated in FIG. 3, other parameters and characteristics of the stimulations can be different as well, including but not limited to, pulse widths, amplitudes, and patterns. The at least one DBS lead 14 can apply the first and second stimulations 22, 26 to locations in a region of the brain. The locations can be the same location, adjacent locations, or proximal locations and one or more contact of the DBS lead 14 can be used. While not shown in FIG. 3, more than one DBS lead 14 may also be used to deliver the first stimulation 22 and the second stimulation 26. The first stimulation 22 has a first frequency (F1) and the second stimulation 26 has a second frequency (F2). The first frequency is an integer multiple of the second frequency, so the second stimulation 26 can be set to occur during a subset of refractory periods of the first stimulation 22. The bubbles surrounding F1 and F2 illustrate examples of sets of neural elements activated by the first and second stimulations 22, 26.

As shown in FIG. 3, panel A, the first stimulation 22 can have a first frequency (F1) and can be applied at a first location in the region of the brain that is adjacent to a second location, and the second stimulation 26 can have a second frequency (F2) and can be applied at the second location. In this example, the first set of neural elements and the second set of neural elements effected by the stimulations are partially overlapping in space, but are theoretically distinct neural populations as described in FIG. 2. As shown in FIG. 3, panel B, the first stimulation 22 can have a first frequency (F1) and is applied at a first location in the region of the brain that is the same as a second location, and the second stimulation 26 can have a second frequency (F2) and is applied at the same, second location. The first set of neural elements is entirely overlapped in space by the second set of neural elements. As shown in FIG. 3, panel C, (the same as panel A) the first stimulation 22 can have a first frequency (F1) and is applied at a first location in the region of the brain that is partially overlapping with a second location, and the second stimulation 26 can have a second frequency (F2) and is applied at the second location. The first set of neural elements is partially overlapping the second set of neural elements. In each of panels A, B, and C the first set of neural elements and the second set of neural elements are independently stimulated.

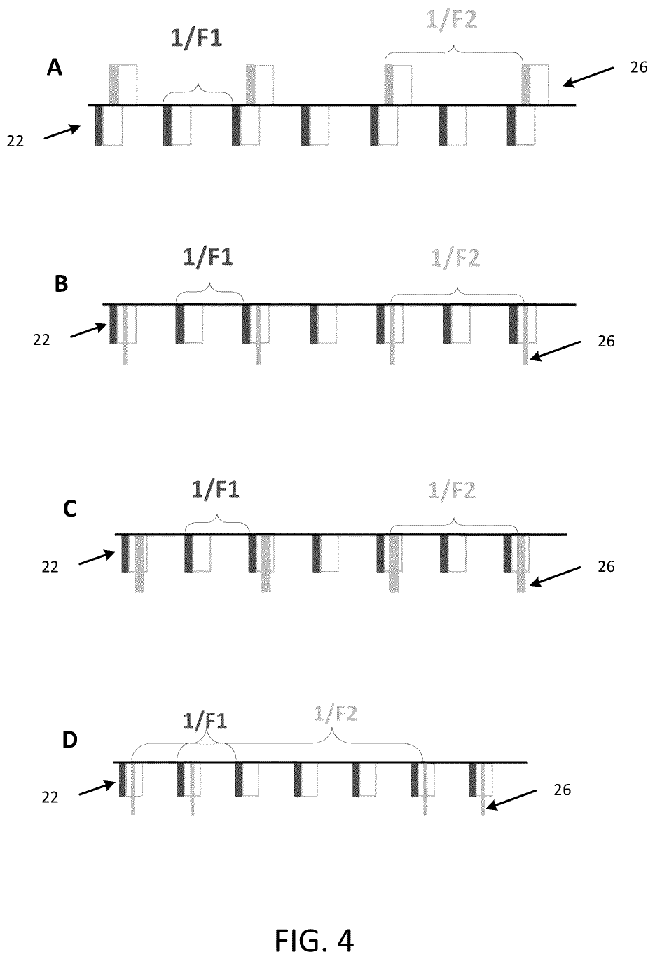

Referring now to FIG. 4, example pulses of the first and second stimulations are shown graphically as pulse trains. In each panel of FIG. 4, the first stimulation 22 is shown first when reading from left to right, but the patterns, frequencies, pulse widths, amplitudes, and polarities can be varied in any combination(s), as long as the pulses of a second stimulation are delivered during the refractory period of a subset of pulses of the first stimulation. The second stimulation 26 is always applied during a refractory period of the first stimulation 22, where the refractory period of the first stimulation is represented as small boxes following each pulse of the first stimulation. For example, FIG. 4, panel A, shows the first stimulation 22 being applied with a first polarity and the second stimulation 26 being applied during the refractory period of every other first stimulation at an opposite polarity, such that the first set of neural elements is stimulated by the first frequency of the first stimulation and then the second set of neural elements are excitable by only the second frequency of the second stimulation. In this example, the first and second stimulations 22, 26 have approximately equal amplitudes and opposite polarities. Additionally, the first frequency can be an integer multiple of the second frequency (where 1/F2 is twice as long as 1/F1). The locations where the stimulations are applied could be the same or different. FIG. 4, panel B, shows the first stimulation 22 having the same polarity as the second stimulation 26, but different amplitudes and pulse widths, where the second stimulation has a larger amplitude and a smaller pulse width than the first frequency. Any combination of larger, smaller, or same amplitudes and/or pulse width are possible. For example, FIG. 4, panel C, shows the first and second stimulations 22, 26 having the same polarity and the second stimulation having a lower frequency than the first stimulation, but the second stimulation having a larger amplitude and pulse width than the second stimulation. FIG. 4, panel D, shows the second stimulation 26 delivered in a doublet pattern, where for every five applications of first stimulation 22 a second stimulation is delivered following the first two applications, but not after the later three applications. It should be noted that any pattern, including an irregular pattern, is possible as long as the pulses of the second stimulation 26 are delivered during a refractory period of the first stimulation 22.

### IV. Methods

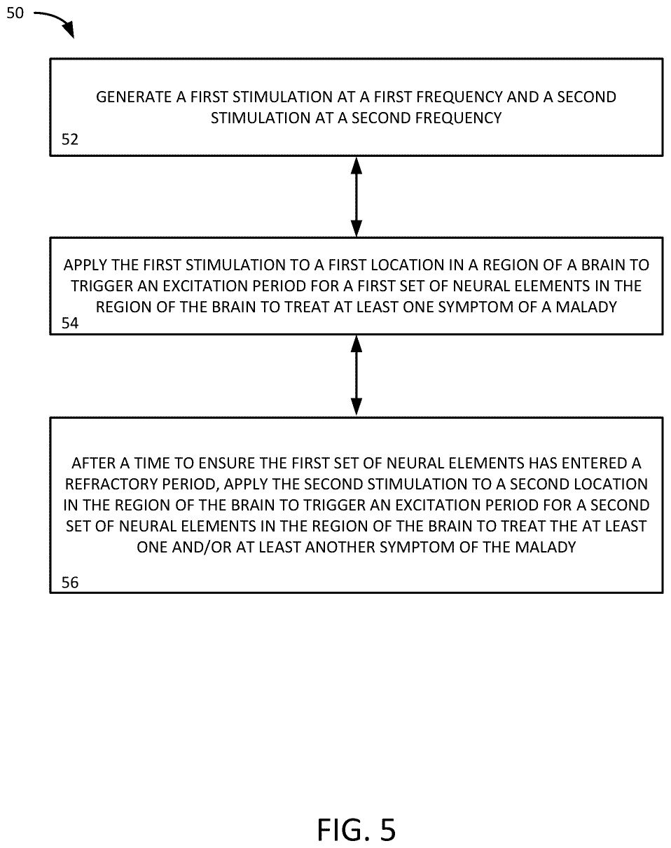

Another aspect of the present disclosure can include methods for selectively stimulating neural sets in a region of the brain, which may treat one or more symptoms of a malady. An example of a method 50 for selective stimulation (using the system of FIG. 1, for example) is shown in FIG. 5 as a process flow diagram with flow chart illustrations. For purposes of simplicity, the method 50 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement method 50.

One or more blocks of the respective flowchart illustrations, and combinations of blocks in the block flowchart illustrations, can be implemented as computer program instructions. The computer program instructions can be stored in memory and provided by a processor of a general purpose computer, special purpose computer, and/or other programmable data processing apparatus to produce a machine (e.g., the signal generator 12), such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The method 50 of the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

Referring to FIG. 5, an aspect of the present disclosure can include method 50 for selective stimulation of sets of neural elements in a region of a brain. At 52, a first stimulation at a first frequency and a second stimulation at a second frequency can be generated by a signal generator. The first stimulation and the second stimulation can have at least one different parameter, such as frequency, pulse width, amplitude, or polarity. The first frequency can be less than 100 Hz and the second frequency can be greater than 100 Hz. The first frequency can also be an integer multiple of the second frequency. The first stimulation can be generated at a first polarity and the second stimulation can be generated at a polarity opposite of the first. At 54, the first stimulation can be applied to a first location in a region of a brain of a patient to trigger an excitation period for a first set of neural elements in the region of the brain to treat at least one symptom of a malady. At 56, after a time to ensure the first set of neural elements has entered a refractory period, the second stimulation can be applied to a second location in the region of the brain to trigger an excitation period for a second set of neural elements in the region of the brain to treat the same symptom (yielding an additive effect) and/or at least another symptom of the malady. The first set of neural elements is not excited by the second stimulation at 56. In one example, the first stimulation can be applied to the first location in the region of the brain at least twice before the second stimulation is applied to the second location.

The first stimulation and the second stimulation can be applied with one or more electrodes, which can be part of a DBS lead having at least one contact. It should be noted that while a DBS lead is described herein, it is not required and any particular grouping of one or more electrodes can deliver the stimulations. If a DBS lead is used, then the at least one DBS lead can be pre-implanted or can be positioned in the region of the brain of the patient for specific application of the first and second stimulations. For example, when one DBS lead is positioned in the region of the brain the DBS lead can include at least one electrical contact to deliver the first stimulation to the first location and the second stimulation to the second location. The first location and the second location can be at least one of proximal, adjacent, overlapping, or identical to one another. The number and types of DBS lead(s)/electrode(s) and/or electrical contacts can be based on the sites of the first and second locations. The first set of neural elements and the second set of neural elements may be at least partially at the first location and the second location, respectively, and/or may be in electrical communication with a neural element at the first location and the second location, respectively. The first set of neural elements and the second set of neural elements can be at least one of overlapping (partially or entirely), contiguous, or proximal to one another, depending on the symptoms to be treated.

In one example, the malady can be Parkinson's disease. Parkinson's disease can cause multiple symptoms, sometimes called cardinal and axial symptoms, that may be treatable by targeting neural elements in the subthalamic neighborhood that may be adjacent or overlapping in a region of the brain with different types of stimulations. For example, cardinal symptoms of Parkinson's diseases that can be treated with a first stimulation at a first frequency (e.g., 100 Hz or greater, 130 Hz or higher, or 185 Hz and higher) can include tremor, rigidity, and bradykinesia, while axial symptoms of Parkinson's disease that can be treated with a second stimulation at a second frequency (e.g., 90 Hz or lower, 75 Hz or lower, 65 Hz or lower, or 60 Hz or lower) can include balance and gait. Thus, treating at least one symptom can be treating at least one of tremor, rigidity, and/or bradykinesia and treat the at least the other symptom can be treating an axial problem, a gait problem and/or a balance problem.

Additionally, the first stimulation and the second stimulation can be modulated such that at least one of a polarity, an amplitude, the first frequency or the second frequency, a pulse-width, and a stimulation location of each of the first stimulation and the second stimulation are different. The modulation can be in response to receiving feedback from one or more sensors (e.g., physiological sensors (ECG, EEG, blood pressure, etc.), movement sensors (accelerometer, IMU, gyroscope, etc.), or feedback directly from the patient to better treat the at least one symptom and/or the at least one other symptom.

From the above description, those skilled in the art will perceive improvements, changes, and modifications. Such improvements, changes and modifications are within the skill of one in the art and are intended to be covered by the appended claims.

1. A method comprising:
generating, by a system comprising a processor, a first stimulation at a first frequency and a second stimulation at a second frequency;
sending, by the system, the first stimulation to an electrode for application to a first location in a region of a brain of a patient to trigger an excitation period for a first set of neural elements in the region of the brain to treat at least one symptom of a malady; and
after a time to ensure the first set of neural elements has entered a refractory period, sending, by the system, the second stimulation to the electrode for application to a second location in the region of the brain to trigger an excitation period for a second set of neural elements in the region of the brain to treat the at least one symptom of the malady and/or at least another symptom of the malady,
wherein the first set of neural elements is not excited by the second stimulation.

2. The method of claim 1, further comprising applying the first stimulation to the first location in the region of the brain at least twice before the second stimulation is applied to the second location.

3. The method of claim 1 wherein the generating the first stimulation and the second stimulation, further comprises:
generating the first stimulation at a first polarity; and
generating the second stimulation at opposite polarity of the first polarity.

4. The method of claim 1, further comprising positioning a DBS lead in the region of the brain of the patient wherein the DBS lead comprises at least one electrode contact to deliver the first stimulation to the first location and the second stimulation to the second location.

5. The method of claim 1, wherein the first location and the second location are proximal to one another or identical.

6. The method of claim 1, wherein the first set of neural elements and the second set of neural elements are at least one of overlapping, contiguous, or proximal to one another.

7. The method of claim 1, wherein the first frequency is less than 100 Hz and the second frequency is greater than 100 Hz.

8. The method of claim 1, wherein the first frequency is an integer multiple of the second frequency.

9. The method of claim 1, wherein the malady is Parkinson's disease, and
wherein the at least one symptom is at least one of tremor, rigidity, and/or bradykinesia, and wherein the at least the other symptom is an axial problem, a gait problem and/or a balance problem.

10. The method of claim 1, further comprising:
modulating, by the system, at least one of a polarity, an amplitude, the first frequency or the second frequency, a pulse-width, and a stimulation location of each of the first stimulation and the second stimulation in response to receiving feedback.

11. A system comprising:
a signal generator configured to generate a first stimulation at a first frequency and a second stimulation at a second frequency; and
at least one DBS lead configured to be positioned within a region of the patient's brain comprising a first set of neural elements and a second set of neural elements, the at least one DBS lead configured to:
apply the first stimulation to a first location in the region of the brain to trigger an excitation period for the first set of neural elements to treat at least one symptom of a malady, and
after a time to ensure the first set of neural elements enters a refractory period, apply the second stimulation to a second location in the region of the brain to trigger an excitation period for only the second set of neural elements to treat the at least one symptom of the malady and/or at least another symptom of the malady,
wherein the first set of neural elements is not excited by the second stimulation.

12. The system of claim 11, wherein the generator is configured to generate the first stimulation at least twice before generating the second stimulation.

13. The system of claim 11, wherein the first stimulation has a first polarity and the second stimulation has an opposite polarity than the first stimulation.

14. The system of claim 11, wherein the at least one DBS lead of the patient is previously implanted in the brain of the patient.

15. The system of claim 11, wherein the first set of neural elements and the second set of neural elements are overlapping.

16. The system of claim 11, wherein the first frequency is less than 100 Hz and the second frequency is greater than 100 Hz.

17. The system of claim 11, wherein the first frequency is an integer multiple of the second frequency.

18. The system of claim 11, wherein the malady is Parkinson's disease, and
wherein the at least one symptom is at least one of tremor, rigidity, and/or bradykinesia, and wherein the at least the other symptom is an axial problem, a gait problem and/or a balance problem.

19. The system of claim 11, wherein the region of the brain is the subthalamic neighborhood.

20. The system of claim 11, wherein the generator is configured to modulate at least one of a polarity, an amplitude, the first frequency or the second frequency, a pulse-width, and a stimulation location of each of the first stimulation and the second stimulation in response to receiving feedback.

21. The system of claim 11, wherein the DBS lead has at least one contact configured to deliver the first stimulation to the first location and at least one contact configured to deliver the second stimulation to the second location.

22. The system of claim 11, wherein the DBS lead has one contact configured to deliver the first stimulation to the first location and the second stimulation to the second location, wherein the first location and the second location are the same location.

23. The system of claim 11, wherein the first set of neural elements and the second set of neural elements are at least one of overlapping, contiguous, or proximal to one another.

24. The system of claim 11, wherein the first location and the second location are proximal to one another or identical.
